(19) United States
(12) Patent Application Publication
(10) Pub. No.: US 2009/0131302 A1
(43) Pub. Date: May 21, 2009

(54) Transient receptor potential vanilloid 1 and uses thereof

(76) Inventors: Pankaj J. Pasricha, Cupertino, CA (US); John H. Winston, League City, TX (US)

Correspondence Address: Benjamin Aaron Adler;ADLER & ASSOCIATES, 8011 Candle Lane, Houston, TX, 77071, US

(21) Appl. No.: 12/082,281

(22) Filed: Apr. 10, 2008

Related U.S. Application Data
(60) Provisional application No. 60/922,931, filed on Apr. 11, 2007.

(51) Int. Cl. A61K 38/00; A61K 31/165; A61P 1/00

(52) U.S. Cl. 514/2; 514/622

(57) ABSTRACT

The present invention describes methods of retarding the development of visceral and somatic hypersensitivities in an individual. Further, the present invention describes a potentially important role for the transient receptor potential vanilloid 1 (TRPV1) in initiation and maintenance of the chronic visceral hypersensitivity and its role in development of irritable bowel syndrome.

### CROSS-REFERENCE TO RELATED APPLICATION

This nonprovisional application claims benefit of priority of provisional U.S. Ser. No. 60/922,931, filed Apr. 11, 2007, now abandoned.

### BACKGROUND OF THE INVENTION

1. Field of the Invention

The present invention relates to the fields of gastrointestinal disorders and the pathogenesis of irritable bowel syndrome (IBS) in humans. Specifically, the present invention describes molecular mechanisms that contribute to long-term visceral hypersensitivity. The present invention also defines an important role for the transient receptor potential vanilloid 1 (TRPV1) in initiation and maintenance of persistent visceral hypersensitivity.

2. Description of the Related Art

Irritable bowel syndrome (IBS) is defined by the occurrence of intermittent periods of abdominal pain and altered bowel habits in the absence of observable biological abnormalities [1, 3]. Although no pathogenic mechanisms have been defined, human studies demonstrate that irritable bowel syndrome is associated with a state of chronic visceral hypersensitivity but the mechanisms responsible for the generation and maintenance of visceral hypersensitivity in patients are not known [4, 7]. A popular theory is that irritable bowel syndrome has its roots early in life with various factors being implicated including psychological stress, parental influence, physical/social abuse, dietary and/or chemical intolerance and infections [8-10]. The diversity of these factors suggest that the early life period may be associated with an inherent predisposition of the gastrointestinal tract and associated nervous elements to develop long-term changes in response to transient stressors. This concept is partly supported by animal models that have utilized either maternal deprivation or inflammation of the colon as initiating events in neonatal animals [11-14].

Previous studies have described an animal model of chronic visceral hypersensitivity based upon mechanical and chemical irritation of the colon of neonatal rats [11]. However, the magnitude of the insults used in that study (balloon distention or mustard oil) were capable of producing robust inflammation and/or injury. The clinical and physiological relevance of such noxious stimulation in the context of human irritable bowel syndrome is questionable.

Recent studies support a role for the vanilloid receptor, transient receptor potential vanilloid 1 (TRPV1) in mechanosensation and in mediating mechanical hypersensitivity of visceral afferents. TRPV1 is a cation channel expressed on the majority of nociceptive sensory afferents that is activated by acid and various noxious and inflammatory stimuli. TRPV1 activation produces mechanical hyperalgesia, and knockout mice exhibit decreased mechanical sensitivity of jejunal and colonic afferents suggesting that TRPV1 regulates sensitivity of visceral nociceptors [15, 16]. TRPV1 antagonist capsazepine inhibited colon afferent fiber responses to stretch [16] and jejunal afferent responses to acid [15]. In the rat, TRPV1 immunoreactivity is detected on 82% of thoracolumbar and 50% of lumbar-sacral colonic dorsal root ganglia (DRG) neurons [17], and is observed in nerve fibers within the myenteric ganglia, within the muscle layers and within the mucosa of the colon [18].

The development of behavioral hypersensitivity in various animal models of nociception correlates with the activation two mitogen activated protein kinases (MAPK) p38 and ERK (extracellular signal regulated kinases, p42 and p44) in dorsal root ganglia neurons and treatment with MAPK inhibitors blocks/attenuates development of behavioral hypersensitivity [90-107]. Mitogen activated protein kinases transduce extracellular stimuli into intracellular alterations in protein activity and transcription, and are activated by phosphorylation of specific residues on each kinase [108, 109]. Brief noxious stimuli produce MAPK activation in dorsal root ganglia neurons within minutes that quickly returns to baseline whereas more persistent models of inflammation or neuropathic pain produce long-term activation [87-107]. Noxious stimuli induce calcium influx leading to activation of ras-mitogen and extracellular signal regulated kinase kinase (MEK)-ERK cascade in dorsal root ganglia neurons [92]. p38 is also activated by calcium influx and by various cytokines and growth factors including nerve growth factor (NGF) [93]. Peripheral administration of TRPV1 agonists (heat, capsaicin) activates p38 and ERK in dorsal root ganglia neurons, and both activated p38 (phosphorylated p38) and activated ERK co-localize with TRPV1 immunoreactivity in dorsal root ganglia neurons. Noxious stimulation of the stomach (with 0.5 M HCl) in rats also leads to increased ERK phosphorylation in TRPV1 gastric spinal afferents in the dorsal root ganglia at 4 and 6 hours.

The potent and selective p38 inhibitor SB-203580 blocks the development of behavioral hypersensitivity in models of inflammation and neuropathy [93, 98, 102]. Similar findings were observed with p38 inhibitors FR167653 [94] and SD-282 [101]. Application of U0126, an inhibitor of MEK, which phosphorylates and activates ERK reduces epinephrine induced mechanical hypersensitivity [92] and attenuates thermal and mechanical hypersensitivity in models of inflammation and neuropathic pain [97, 106]. No studies have directly linked MAPK activation to functional changes in DRG neurons.

Brain derived neurotrophic factor or BDNF, expressed by NGF responsive nociceptor neurons, is a neurotransmitter of considerable importance in maintaining sensitization. Brain derived neurotrophic factor is expressed by 20-30% of all dorsal root ganglia, predominantly in small to medium sized neurons where it is co-expressed with other neuropeptides such as CGRP (110). Its expression is significantly enhanced in models of inflammatory pain (111). Brain derived neurotrophic factor is expressed and released from the spinal terminals of the primary afferents and acts on its high affinity receptor, TrkB, to sensitize the response of lamina II neurons to high threshold primary afferent inputs via effects that include NMDA receptor involvement and potentiation of synaptic efficacy (112). A potential role for brain derived neurotrophic factor has also been identified in colonic pain, adding to the significance of examining the role for this molecule in development of persistent visceral hypersensitivity (113). The contribution of MAPK and brain derived neurotrophic factor to the development of persistent visceral sensitization and to sensitization of colon dorsal root ganglia neurons is not known.

The prior art is deficient in the knowledge of long-lasting changes in visceral sensitivity in response to the phenomenon of neonatal vulnerability to colonic sensitization. Further, the prior art also lacks the knowledge of the molecular basis for the induction of as well as maintenance of this sensitization, and the role of the vanilloid receptor, transient receptor potential vanilloid 1 (TRPV1) in persistent visceral hypersensitivity.

### SUMMARY OF THE INVENTION

Irritable bowel syndrome is defined by the occurrence of intermittent periods of abdominal pain and altered bowel habits and the absence of observable biological abnormalities. No pathogenic mechanisms have been defined, and little is known about underlying molecular changes responsible for symptom generation. Human studies demonstrate that irritable bowel syndrome is associated with a state of chronic visceral hypersensitivity suggesting that processing of visceral sensory information is altered, but the mechanisms responsible for the generation and maintenance of visceral hypersensitivity in patients are not known. To gain insight into the etiopathogenesis of visceral hypersensitivity in irritable bowel syndrome, the present invention has developed a rat model based upon neonatal sensitization by a noxious stimulus delivered to the colon.

Hence, the present invention is directed to a method of retarding the development of visceral hypersensitivity in an individual in need of such treatment comprising administering to the individual a pharmacologically effective amount of a transient receptor potential vanilloid 1 (TRPV1) antagonist.

The present invention is also directed to a method for ameliorating irritable bowel syndrome in an individual in need of such treatment comprising; administering a pharmacologically effective amount of a transient receptor potential vanilloid 1 (TRPV1) antagonist.

The present invention is further directed to a method of reducing sensitization of Dorsal root ganglion neurons in the colon of an individual in need of such treatment comprising; administering a pharmacologically effective amount of a transient receptor potential vanilloid 1 (TRPV1) antagonist.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1D show effects of neonatal P10 acetic acid treatment on sensitivity to CRD when measured at 8 weeks of age. Abdominal withdrawal reflex scoring criteria (FIG. 1A): abdominal withdrawal reflex scores as a function of distention pressure, P10 saline treated, n=9; P10 acetic acid treated, n=8. ***p=0.009, **p=0.023, ^{#}p=0.014, ^{##}p=0.03 (FIG. 1B): EMG activity in the external oblique muscle in response to graded CRD (FIG. 1C): EMG is expressed in units of V·sec; each point represents the mean of 17 rats from 3 independent experiments (neonatal treatment, F=5.288, p=0.030; distention pressure, F 3.38.2 p<0.001, two way repeated measures ANOVA). **p=0.010, *p=0.035. Fecal pellet output in adult rats expressed as percent controls *p<0.05.

FIG. 2 shows effect of colonic treatment with 5% acetic acid at 8 weeks on sensitivity to CRD at 12 weeks. Abdominal withdrawal reflex scores as a function of distention pressure, n=5. A significant increase in abdominal withdrawal reflex grade in response to increasing CRD pressure was observed in both P10 acetic acid and P10 saline treated rats, p<0.001, respectively. Mann-Whitney rank sum test at each distention pressure found no significant differences between treatment groups (50 mm Hg, p=0.690, 60 mm Hg, p=0.421, 70 mm Hg, p=0.310, 80 mm Hg, p=0.69).

FIGS. 3A-3J show effect of P10 acetic acid treatment on colons of neonatal and adult rats. (FIG. 3A-FIG. 3D): Photomicrographs of H&E stained sections from colons of neonatal rats: saline (FIG. 3A, FIG. 3C) and acetic acid (FIG. 3B, FIG. 3D) treated at 4 hours (FIG. 3A, FIG. 3B) and (FIG. 3C, FIG. 3D) 24 hours after treatment. (FIG. 3E-FIG. 3J): Photomicrographs of H&E stained sections from proximal (FIG. 3E, FIG. 3F), middle (FIG. 3G, FIG. 3H) and distal colon (FIG. 3I, FIG. 3J) from P10 saline (FIG. 3E, FIG. 3G, FIG. 3I) and P10 acetic acid (FIG. 3F, FIG. 3H, FIG. 3J) treated rats.

FIG. 4 shows the effect of neonatal P10 capsaicin treatment on sensitivity to CRD when measured at 7 weeks of age. Neonatal treatment with capsaicin resulted in increased sensitivity of these rats to CRD compared to vehicle treated or unmanipulated rats (n=5). *p=0.007, **p=<0.001

FIGS. 5A-5B show effects of pretreatment with TRPV1 antagonist SB-366,791 (1.3 mg/kg s.c.) prior to acetic acid infusion in 10 days old pups on the sensitivity of these rats to graded CRD when they were tested at eight weeks of age (n=10). FIG. 5A: abdominal withdrawal reflex scores as a function of distention pressure, P10 acetic acid vehicle vs P10 acetic acid SB-366,791: *p<0.05. P10 acetic acid vehicle vs P10 saline vehicle, #p<0.05. FIG. 5B: EMG activity (V sec) in the external oblique muscle in response to graded CRD. P10 acetic acid vehicle vs P10 acetic acid SB-366,791: ***p=0.002, *p=0.034, **p<0.03. P10 acetic acid vehicle vs P10 saline vehicle: +p=0.025, ++p<0.001.

FIGS. 6A-6C show pretreatment with TRPV1 antagonists SB-366,791 (2.6 mg/kg s.c.) A, B or I-RTX, 2 mmol/kg C, D prior to acetic acid infusion in 10 days old pups reduced sensitivity of these rats to graded CRD when tested at eight weeks of age (n=10). FIG. 6A. SB-366,791, abdominal withdrawal reflex grade: ****p=0.001, ***p=0.002, **p=0.003, *p=0.014, FIG. 6B. SB-366,791, EMG: *p=0.050, **p=0.038, ***p=0.003 FIG. 6C. I-RTX, abdominal withdrawal reflex grade: ***p=0.002, **p=0.037, *p=0.05 D. I-RTX, EMG: ###p<0.001, ##p=0.003, #p=0.047.

FIG. 7 shows treatment with TRPV1 antagonist SB-366791 reduced sensitivity to CRD in P10 acetic acid treated rats when tested at 7-8 weeks of age (n=10). P10 acetic acid treated with SB-366,791 compared to P10 acetic acid rats treated with vehicle, *p=0.023, **p=0.021. P10 acetic acid treated with vehicle compared to P10 saline treated with vehicle, *p=0.042, **p=0.009.

FIGS. 8A-8D TRPV1 expression in adult rats. Western blots showing TRPV1 protein expression in lumbar sacral (LS) DRG (L6, S1) and thoracolumbar (TL) DRG (T13, L1, L2) of adult rats, **p<0.01, *p<0.03 (FIG. 8A). Western blots showing TRPV1 protein expression in lumbar sacral (LS) and thoracolumbar (TL) spinal cord of adult rats (FIG. 8B). TRPV1 immunofluorescence (green) in DiI (red) labeled colon afferents in sections from LS and TL DRG (FIG. 8C). Hollow arrows designate neurons showing co-localization between TRPV1 and DiI; thin arrows mark DiI labeled neurons in which TRPV1 immunofluorescence was not detected. Bar graphs showing fold change in TRPV1 mRNA in TL and LS DRG in adult rats that were neonatally sensitized with acetic acid compared to controls, *p<0.05 (FIG. 8D).

FIG. 9A-9F show the excitability of LS and TL colon DRG neurons from 8 weeks old rats treated with saline or acetic acid as neonates. Action potential at rheobase in neuron from control, (FIG. 9A), and acetic acid treated rats (FIG. 9B). (FIG. 9C), Average rheobase Action potentials elicited at 2× the rheobase in neurons from control, (FIG. 9D), and acetic acid treated rats, (FIG. 9E). (FIG. 9F), Average number of action potentials elicited by current injection at 2× the rheobase. Neurons were isolated from 10 control and 10 P10 AA rats: TL control, n=18, TL AA, n=25, LS control, n=32, LS AA, n=27

FIG. 10 shows TRPV1 and BDNF mRNA expression in S1 DRG, *P<0.02 *p<0.03.

FIG. 11 shows isolation of Fluorogold labeled colon DRG neurons by laser capture microdissection, (FIG. 11A). S1 DRG section (FIG. 11B, FIG. 11C) Fluorogold labeled neuro before and after LCM. Quantitative RT-PCR (FIG. 11D).

FIG. 12A-12B shows Western blots with phosphorylated MAPK in extracts prepared one hour after colon infusion from pooled DRG from LS (lumbar/sacral, L6,S1) TL (thoracolumbar, L1-L3) and CE (cervical, C5-C8) DRG from 5 pups were pooled for each sample (UM, unmanipulated, SAL, saline, AA, acetic acid). P-p38 expression is calculated relative to UM controls at each spinal level.

### DETAILED DESCRIPTION OF THE INVENTION

The present invention has successfully induced long-lasting visceral sensitization with a relatively innocuous stimulus (dilute acetic acid) that appears to cause little or no overt injury or inflammation following administration in the neonatal period. As adults (8-12 weeks) these rats displayed increased sensitivity to colorectal distention in the absence of histological evidence of inflammation or other anatomical abnormalities. This persistent visceral hypersensitivity was significantly attenuated both by pre-treatment with TRPV1 antagonists in the neonatal period and by TRPV1 antagonism in hypersensitive adults suggesting a role for this channel in the generation and maintenance of persistent visceral hypersensitivity even in the absence of observable abnormalities. These phenomena appear to resemble findings in IBS patients where a majority of patients exhibit chronic visceral hypersensitivity as assessed by colorectal distention (CRD) [4, 32-37], without overt inflammation.

Although both the etiopathogenesis of colonic sensitization in this model and its biological basis are unclear, this study provides important new insight. The data demonstrate that even relatively minor degrees of noxious stimulation in the neonatal period can produce long-term sensitization without an initial period of overt injury or inflammation. In adults, intracolonic instillation of acetic acid at a much higher (4%) concentration than used in the current model (P10 0.5% acetic acid) causes inflammation and can sensitize pelvic nerve afferents, but is not known whether these effects are due to inflammation per se or to a direct effect of acetic acid itself [38, 39]. Burton and Gebhardt detected sensory effects prior to detecting inflammation suggesting possible effects of acetic acid on sensory nerves or other cell types independent of activation of inflammatory pathways in the intestine [38]. In the model, dilute acetic acid treatment of P10 pups did not cause a major inflammatory response in the colon of either the pups (at 4 & 24 hours) or at 8 weeks. Stress, per se, is also unlikely to be a major factor as control animals were handled and manipulated in a similar fashion to the sensitized rats.

It is possible that these results do not reflect direct activation of TRPV1 and could be explained by acetic acid causing local release of one or more pro-algesic substances such as NGF [40], serotonin [41], anandemide [42], ATP [43] or leukotrienes [44, 45] that can sensitize/activate TRPV1. Although no overt inflammation was detected in the neonatal period following acetic acid administration as judged by histological examination and MPO assays, detailed studies assessing the levels of these substances in neonatal colons following acetic acid administration may be useful. However, the fact that capsaicin by itself can also lead to a persistent visceral hypersensitivity strongly implicates a direct role for TRPV1.

These results are also consistent with recent studies on TRPV1 knockout mice showing that TRPV1 is required for mechanical sensitization (but not activation) of stretch sensitive muscular colonic afferent fibers by an acidic (pH 6.0) inflammatory soup [16]. This study also showed that Acid sensing ion channel 3 (ASIC3), another channel also activated by acid is implicated in sensitization of stretch-sensitive colonic afferents [16]. Future studies using specific inhibitors for this channel would address its role in initiating sensitization.

This data showing that specific antagonism of TRPV1 in the neonatal period prevents the development of persistent visceral hypersensitivity in adults strongly suggests that activation of TRPV1 may represent one of the initial molecular events in the development of persistent visceral hypersensitivity. Since TRPV1 is activated by pH<6.0, it is possible that events in the colon during the neonatal period such as changes in colonic pH due to carbohydrate malabsorption, infections and food allergies could produce direct activation of TRPV1 that may contribute to visceral hypersensitivity. This phenomenon may explain the development of irritable bowel syndrome even in the absence of a history of a significant gastrointestinal infection. Further because of its ability to be activated by several types of noxious stimuli produced during tissue injury, including heat, local tissue acidosis, and several pro-algesic metabolites, this receptor could potentially represent a common link for a variety of other sensitizing events in the period of neuronal vulnerability. Thus, although initiated by an acidic stimulus that may directly activate TRPV1, this model has great potential relevance to development of visceral hypersensitivity in humans. Since the stress of maternal deprivation also triggers long-term changes in visceral sensitivity [12, 13], TRPV1 inhibition may affect either the development or the maintenance of hypersensitivity in this model.

Although the importance of their relative roles remains controversial, there is evidence for both central and peripheral sensitization in clinical and experimental forms of irritable bowel syndrome. Electrophysiological studies of dorsal roots showing sensitization of colonic afferent nerves in a related model of chronic visceral sensitization initiated by neonatal mechanical irritation is consistent with the hypothesis that TRPV1 mediated sensitization of peripheral nerves contributes to the generation of persistent visceral hypersensitivity [46]. However, since TRPV1 is also expressed on afferent nerve endings in the spinal cord and in other sites in the brain central effects cannot be ruled out. The extent to which the antagonists used penetrate the blood brain barrier is not known although the ability of SB-366791 to block capsaicin effects on thermoregulation is indicative of central effects [25].

The results also provide important insight into the role of TRPV1 in the maintenance of colonic hypersensitivity. The molecular basis for this hypersensitivity in particular or for noxious mechanical sensory transduction from the viscera in general, is not known. In hypersensitive adults, treatment with the specific TRPV1 antagonist SB-366791 reduced behavioral response to colorectal distention in sensitized rats. Although this is the first study of its kind, other investigators have shown that TRPV1 knockout mice exhibit decreased VMR responses to colorectal distention [16], and the TRPV1 antagonist capsazepine reduces jejeunal [15] and colonic afferent responses to distention [16]. These results are consistent both with a direct role of TRPV1 in transducing mechanical stimuli and/or a role in regulating the mechanosensitivity of visceral sensory neurons. These findings contrast with somatic pain models in which mechano-sensation is not lost in TRPV1 knockouts suggesting that TRPV1 does not contribute to somatic mechano-sensation [47, 48]. Although previous studies show that administration of TRPV1 antagonists reduce behavioral hyperalgesia in visceral inflammatory pain models in the urinary bladder [49, 50] and colon [51] and in bone cancer pain [52], this is the first study showing a potential role for TRPV1 in maintaining hypersensitivity to colonic distention in a model of functional bowel pain and suggests a novel therapeutic target for conditions such as irritable bowel syndrome.

The fact that TRPV1 antagonism does not alter the response to colorectal distention in controls suggests that the drug does not act as a general analgesic but dampens a pathologically heightened response of a sensitized nociceptive system. This effect may be due to an increase in TRPV1 expression or function or both. An increase in TRPV1 protein expression was observed in both LS and TL DRG containing colon afferents in sensitized adults, and an increase in the numbers of TL and LS colon afferents expressing detectable levels of TRPV1 immunofluorescence. Up-regulation at the mRNA level was observed in LS but not TL DRG suggesting that different regulatory mechanisms may be operating in LS and TL afferents. Since LS and TL afferents differ in their neurochemical coding and range of physiological responses to distention, [17] they may also differ in their regulation of TRPV1 expression. Increased expression of TRPV1 is observed in animal models of inflammatory pain including acid insult to the gastric mucosa in rats [53], osteoarthritis [54] and CFA-induced inflammation [55, 56]. In humans, TRPV1 expression is increased in conditions such as mastalgia [57], vulvodynia [58], human tooth pulp with caries [59], esophageal pain [60], rectal pain [61] as well as intestinal inflammation [62, 63]. In patients with severe rectal hypersensitivity it has been shown that TRPV1 levels are increased in the rectal mucosa [64]. Future studies will establish whether this increase in TRPV1 protein levels leads to increased colon afferent sensitivity to the TRPV1 agonist capsaicin and/or to other potential physiological mediators such as serotonin or mast cell tryptase that can activate and/or sensitize TRPV1 [65, 66].

A persistent increase in TRPV1 responsiveness in adulthood could arise from continued exposure to various types of sensitizing factors generated by immune activation [67-72]. Colonic hypersensitivity in both humans and in various animal models is associated with identifiable changes in gut neuroimmune function such as altered cytokine levels [67-70], increased expression of nerve growth factor [71], increased paracellular permeability [72], and increased mast cell numbers 173-751. These initial findings failed to show significant ongoing inflammation, increases in mast cell density in adults or changes in a panel of inflammatory cytokines with the exception of a small but significant increase in INF-gamma expression in the proximal colon. It is possible that this increase may be indicative of subtle immune changes that may contribute to the persistence of hypersensitivity. In a rat model of visceral hypersensitivity in the absence of overt inflammation induced by chronic water avoidance stress, a 3 fold increase in IFN-gamma mRNA was observed in the colon [76]. Increased IFN-gamma induced by stress is linked to increased intestinal permeability [77, 78]. These studies are consistent with an indirect role of persistent IFN-gamma expression in the perpetuation of hypersensitivity via mediating increases in intestinal permeability. These findings do not rule out the possibility that as yet undetected changes in gut immune function (other than IFN-g) and paracellular permeability may occur in this model and contribute to the development of hypersensitivity.

The data do not exclude the possibility that other channels contribute to sensitization in this model. In addition to TRPV1, several other channels expressed on colon sensory neurons, ASIC3, the ATP activated ion channel P2X3, and the N-methyl D-aspartate receptor [79] have been proposed to participate in mechanotransduction. ASIC3 knockout mice also display reduced VMR in response to colorectal distention and reduced responses of afferent nerve fibers to circumferential stretch [16, 80]. An increased distension-evoked release of ATP as well as an increase in the number of DRG neurons supplying the colorectum expressing P2X3 receptors was observed in a rat model of colitis, and ATP increases distention evoked responses of pelvic nerve afferents [81]. In the bladder, P2X3 is activated by release of ATP from cells adjacent to nerve endings following mechanical stimulation [82] and TRPV1 is required for ATP release from bladder epithelium [83] suggesting a potential functional link between these two channels.

Another important lesson from this model is the unique vulnerability of the immature animal to a common but transient colonic insult such as acidification of colonic contents. Adults treated with acetic acid were not hypersensitive when tested after 4 weeks, consistent with previous results [11]. It is known that the extrinsic nervous system may be vulnerable to alteration by strong stimuli in the neonatal period, perhaps due to immaturities in regulatory systems such as the hypothalamic-pituitary axis, and intrinsic pain inhibitory systems [84, 85]. It is also possible that genetically determined persistence of this vulnerability may predispose to sensitization even in some adults. Identification of factors contributing to neonatal vulnerability in this model therefore may be relevant to the development of IBS in general as it may provide insight into why only a small percentage of patients develop IBS following infectious diarrhea [86], or other triggering events.

In conclusion, the present invention described a new model for persistent colonic sensory dysfunction following a transient noxious stimulus in the neonatal period that has the potential for providing unique insight into the pathogenesis of irritable bowel syndrome. These results show that prophylactic treatment with two different TRPV1 antagonists SB-366791 and I-RTX in neonates prevents the development of sensitization in adults. Further, once sensitization has developed it can also be ameliorated by treatment with a TRPV1 antagonist. These results suggest an important role for TRPV1 in irritable bowel syndrome and persistent colonic hypersensitivity.

In one embodiment of the present invention there is provided a method of retarding the development of hypersensitivity in an individual in need of such treatment consisting of administering to the individual a pharmacologically effective amount of a transient receptor potential vanilloid 1 (TRPV1) antagonist. In general, the hypersensitivity is visceral or somatic. Specifically, the visceral hypersensitivity is due to mechanical, chemical or thermal stimuli. In addition, the hypersensitivity is present in the absence of overt inflammation or peripheral pathology. In general, the transient receptor potential vanilloid 1 antagonist is SB-366791. Additionally, the transient receptor potential vanilloid 1 antagonist is I-RTX. In general, the transient receptor potential vanilloid 1 antagonist attenuates excitability of colon DRG neurons. Specifically, the attenuation of the excitability of colon DRG neurons is due to inhibition of the MAPK pathway. In general, the individual suffers from the irritable bowel syndrome, functional dyspepsia, visceral hyperalgesia, visceral allodynia or altered gastric motility.

In another embodiment of the present invention there is a method for ameliorating irritable bowel syndrome in an individual in need of such treatment consisting of administering a pharmacologically effective amount of a transient receptor potential vanilloid 1 (TRPV1) antagonist. Specifically, the transient receptor potential vanilloid 1 antagonist is SB-366791. Alternatively, the transient receptor potential vanilloid 1 antagonist is I-RTX. In general, the transient receptor potential vanilloid 1 antagonist attenuates excitability of colon DRG neurons. Specifically, the attenuation of the excitability of colon DRG neurons is due to inhibition of the MAPK pathway. The individual suffers from visceral hyperalgesia, visceral allodynia, abdominal bloating and altered gut motility.

In yet another embodiment of the present invention, there is provided a method of reducing sensitization of the colon dorsal root ganglion neurons in an individual in need of such treatment consisting of administering a pharmacologically effective amount of a transient receptor potential vanilloid 1 (TRPV1) antagonist. Specifically, the sensitization leads to the development of chronic visceral hypersensitivity. Moreover, the chronic visceral hypersensitivity is present in the absence of any overt inflammation or peripheral pathology. In general, the sensitization of the colon dorsal root ganglion neuron is due to early life environmental effects, stress, psychologic state and genetic factors. Specifically, the transient receptor potential vanilloid 1 antagonist attenuates excitability of colon DRG neurons. The attenuation of the excitability of colon DRG neurons is due to inhibition of the MAPK pathway. Specifically, a representative transient receptor potential vanilloid 1 antagonist is SB-366791. Alternatively, the transient receptor potential vanilloid 1 antagonist may be I-RTX. In general, the individual suffers from the irritable bowel syndrome, functional dyspepsia, visceral hyperalgesia, visceral allodynia or altered gastric motility.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Animals

Male Sprague Dawley rats were used in all experiments. The Institutional Animal Care and Use Committee, University of Texas Medical Branch approved procedures performed on animals.

### EXAMPLE 2

### Colonic Sensitization

For neonatal sensitization, rats were obtained as litters of 4 days old pups. Ten day old rat pups (P10) received an infusion of 0.2 ml of 0.5% acetic acid solution in saline into the colon 2 cm from the anus and, controls received an equal volume of saline. To treat neonatal rats with capsaicin, ten day old rat pups received an infusion of 0.2 mls of 50 mg/ml capsaicin or 0.2 mls vehicle: 10% ethanol, 10% Tween 20, 80% saline or were left unmanipulated. Visceral sensitivity and inflammatory parameters were measured in these rats between 8 and 12 weeks of age. For adult sensitization, eight weeks old rats were administered 1 ml each of a 5% solution of acetic acid in saline into the descending colon; controls received saline. After 4 weeks, rats were tested for sensitivity to colorectal distention.

### EXAMPLE 3

### Visceral Sensitivity

In rats, high intensity colorectal distention represents a noxious visceral stimulus that produces both aversive behaviors and visceromotor responses (contraction of the abdominal and hindlimb muscles) that are quantifiable and reproducible measures of acute visceral pain.¹⁹ These effects are also observed in humans where colorectal pain is accompanied by cardiovascular and respiratory reflexes and by increases in the tension of abdominal wall muscles.²⁰ In initial studies, we used a grading system based upon the abdominal withdrawal reflex (AWR) as well as a measure of the electromyographic activity (EMG) of the external oblique muscle. The abdominal withdrawal reflex represents a characteristic set of behaviors produced by involuntary motor reflexes that are activated by colorectal distention. Visceral hypersensitivity was measured by grading the response of rats to colorectal distention as previously described.¹¹ Briefly, under mild sedation with 1% Brevital (Eli Lily & Co Indianapolis, Ind.) 25 mg/kg i.p.), a flexible balloon (5 cm) constructed from a surgical glove finger attached to a tygon tubing was inserted 8 cm into the descending colon and rectum via the anus and held in place by taping the tubing to the tail. Rats were placed in small Lucite cubicles (20×8×8 cm) (Bioengineering Department, UTMB) and were allowed to adapt for 30 minutes. CRD was performed by rapidly inflating the balloon to constant pressure. Pressure was measured using a sphygmomanometer connected to a pressure transducer. The balloon was inflated to various pressures: 10, 20, 30, 40, 50, 60, 70 & 80 mm Hg, for a 20 seconds stimulation period followed by a 2 minute rest. Behavioral responses to colorectal distention were measured by visual observation of the abdominal withdrawal reflex by blinded observer and the assignment of an AWR score as follows: 1=Normal behavior without response 2=Contraction of abdominal muscles; 3=Lifting of abdominal wall; 4=Body arching and lifting of pelvic structures.

To obtain electromyographic (EMG) measurements of visceromotor responses, under anesthesia (Nembutal, 50 mg/kg i.p.), two electrodes were implanted in the external oblique muscle and externalized behind the head. Rats were allowed one week to recover from the surgery. colorectal distention was performed as described above with 20 seconds of distention followed by two minute rest between distensions of 20, 40, 60 and 80 mm Hg. EMG was recorded continuously during the experiment on a Biopac Systems EMG 100 C, MP100A-CE (Biopac Systems, Inc., Santa Barbara, Calif.). The EMG signal was amplified, filtered at 300 Hz and digitized using Acknowledge (Biopac Systems, Inc.). The area under the curve (AUC) for the EMG signal (during each 20 seconds of distention plus 10 seconds post distention period (a total of 30 seconds) was calculated using an in-house written computer program. The net value for each distension was calculated by subtracting the baseline value derived from the average AUC (30 seconds interval) for the two minutes pre-distention period.

### EXAMPLE 4

### Evaluation of the Colon for Inflammation/Damage

Colons were divided into proximal, middle and distal segments; portions of each segment were placed in 10% buffered formalin and portions were frozen in liquid nitrogen. Haematoxilin & Eosin stained parafin sections were scored for inflammation by a pathologist as described.²¹ Myeloperoxidase (MPO) assays were performed as described.²² Protein was measured by the BCA method (Pierce, Rockford, Ill.). Activity was expressed as the change in absorbance/min/mg protein. Cytokine levels were measured in colon extracts using the Biorad Bioplex assay procedure and normalized to protein.

### EXAMPLE 5

### TRPV1 Inhibitors

SB-366791 N-(3-Methoxyphenyl)-4-chlorocinnamide was purchased from Biomol (Plymouth Meeting, Pa.) and 5′Iodoresiniferatoxin (1-RTX) from Tocris (Ellisville, Mo.). Stock solutions were prepared in DMSO and then diluted in vehicle: 1×PBS, 5% Tween 80, 5% DMSO. SB-366791 is a competitive antagonist of capsaicin with a Ki=18 nM, and it blocks acid-mediated activation.²³⁻²⁵ It exhibited little or no effect at 1 mM in vitro binding assays against 50 receptors of various types or on voltage-gated Ca++ currents or the hyperpolarization activated current in rat DRG neurons, and no effects on AMPA or NMDA mediated hippocampal synaptic transmission.²³ I-RTX specificity has been demonstrated in vitro and it is effective in blocking TRPV1-mediated effects in several animal models in vivo.²⁷⁻³⁰ Both I-RTX and SB-366791 are more potent and selective antagonists than the traditional TRPV1 antagonist capsezapine which has poor pharmacokinetic properties and blocks calcium channels at high concentrations.³¹

### EXAMPLE 6

### TRPV1 Inhibitor Experimental Protocols

In the first experiment, 3 groups of 10 neonatal rats were used. While Group 1 received SB-366791, 1.3 mg/kg subcutaneously 30 minutes prior to colonic acetic acid administration. Group 2 received vehicle subcutaneously prior to colonic acetic acid administration, and Group 3 received vehicle prior to colonic saline administration. In the second experiment, 3 groups of 10 neonatal rats were used. All rats received a drug or vehicle subcutaneously 24 hours and 30 minutes prior to colonic acetic acid administration. Group 4 received SB-366791, 2.6 mg/kg, Group 5 received I-RTX, 2 mg/kg, and Group 6 received vehicle. For both experiments, at 8 weeks of age, EMG recordings and abdominal withdrawal reflex grades were simultaneously obtained in response to graded colorectal distention. To test the effect of TRPV1 inhibitors on adults, 2 groups of 10 rats were used: on post-natal day 10, Group 7 received colonic acetic acid, Group 8 received colonic saline. At 7 weeks of age, Groups 7 and 8 were split: half of each group received SB-366791, 1.3 mg/kg, and half vehicle, subcutaneously 30 minutes prior to colorectal distention. After one week of period, vehicle and drug groups were crossed-over and sensitivity to colorectal distention was measured again.

### EXAMPLE 7

### TRPV1 Expression and Western Blot

Tissues for western blot or RNA extraction were immediately frozen on dry ice and were stored at −80 until further analysis. Tissues were homogenized in extract buffer; 1% NP40, 10% glycerol, 20 mM Tris HCl pH 74, 100 mM NaCl, 50 mM NaF, 2 mM EDTA, 1 mM EGTA, 25 mM b-glycerophosphate, 2 mM sodium pyrophosphate, 1 mM sodium orthovanadate, 1 mM phenylmethylsulfonyl fluoride and Protease Inhibitor Cocktail, 1/100 (Sigma). Proteins were resolved on 7.5% SDS-PAGE gels, transferred to PVDF membranes (BioRad), and incubated with an anti-TRPV1 antibody (PC420, Invitrogen) at 1/500. Bound antibodies were detected by ECL kit (Amersham Pharmacia Biotech, Piscataway, N.J.). The membranes were exposed to X-ray films and subsequently stripped and re-probed with a b-III-tubulin antibody (1/1000, Promega). Films were converted to TIFF image files using a HP scanner, and the TRPV1 and b-III-tubulin bands were quantified using OptiQuant software (Packard). Data was expressed as the ratio of TRPV1 to b-III-tubulin band intensity.

### EXAMPLE 8

### Quantitative RT-PCR

RNA was extracted from frozen DRG using Triozol (Invitrogen). Starting with 1 mg of RNA, two-step quantitative RT-PCR was performed using Applied Biosystems reagents and kits. The following forward and reverse primer and probe sequences were used: TRPV1: GCAAGAAGCGCCTGACTGA (SEQ ID NO: 1), TGAGCATGGCTTTTAGCAGACA (SEQ ID NO: 2), TTTCCTGTCTCTGGGTCTTTGAACTCGCT (SEQ ID NO: 3); b-III-tubulin: GGGCCTTTGGACACCTATTCA (SEQ ID NO: 4), GCCCTTTGGCCCAGTTGT (SEQ ID NO: 5), CCTGACAACTTTATCTTCGGTCAGAGTGGTG (SEQ ID NO: 6). TAQMAN PCR was performed on an Applied Biosystems 5700. Relative changes in TRPV1 mRNA levels were calculated by the DDCt method using b-III-tubulin as a normalizer. No RT reactions were performed as controls.

### EXAMPLE 9

### Immunofluorescence

The lipid soluble fluorescent dye, DiI (1,1′-dioleyl-3,3,3′,3-tetramethylindocarbocyanine methanesulfonate, Invitrogen), 25 mg in 0.5 ml methanol, was injected in 2 μl volumes at 8-10 sites on the exposed colon from the level of the bladder to 6 cm oral. After one to two weeks, rats were perfused transcardially with 150 ml phosphate buffered saline (PBS) followed by 400 ml ice cold 4% paraformaldehyde in PBS. Dorsal root ganglia, T12 to S2 were removed and were post-fixed one hour in PFA and cryoprotected overnight in 20% sucrose in PBS. 10 micron sections on plus slides were incubated sequentially with TRPV1 antibody (PC420, Invitrogen, 1/200) and then with anti-rabbit Alexa Fluor 488 (Invitrogen, 1/200). Sections were viewed using a BX60 Olympus microscope equipped with filter cubes appropriate for DiI (Rhodamine filter) and appropriate band pass and barrier filter for Alexa-488. Images were captured and were analyzed using Metaview software. To ensure that a neuron is counted only once, serial sections were placed on consecutive slides with at least 50 microns between sections on the same slide.

### EXAMPLE 10

### Statistics

Data is expressed as mean+/−standard error of the mean (SE). Analyses were performed on Sigma Stat software (Jandel, SPSS Science, Chicago, Ill.). For AWR behavioral grades, a Friedman ANOVA was used to determine whether scores changed with pressure within each experimental group. Median AWR scores at each distention pressure were compared between treatment groups by a Mann-Whitney rank sum test. EMG was analyzed by two way repeated measures ANOVA with distention pressure as the repeated measure. MPO data was analyzed by t-test or one-way ANOVA where appropriate. In all analyses, p<0.05 was taken as significant.

### EXAMPLE 11

### Acetic Acid Treatment of Colon of P10 Rats Produces Persistent Visceral Hypersensitivity

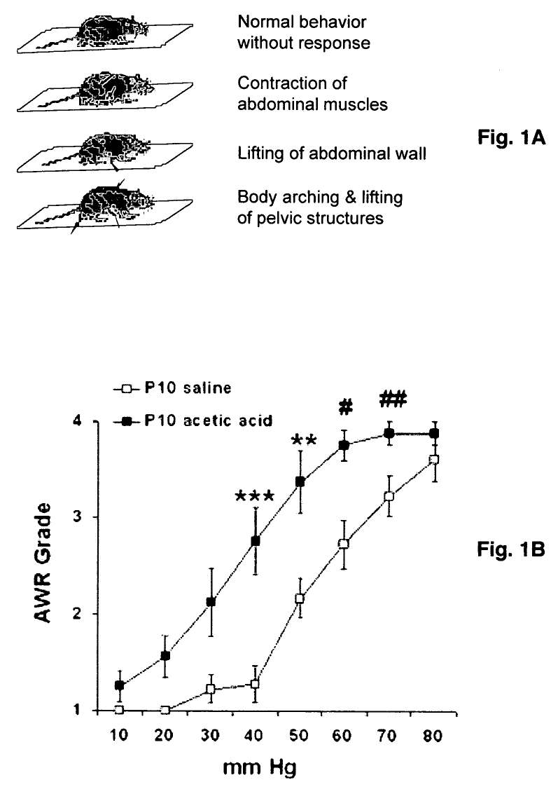

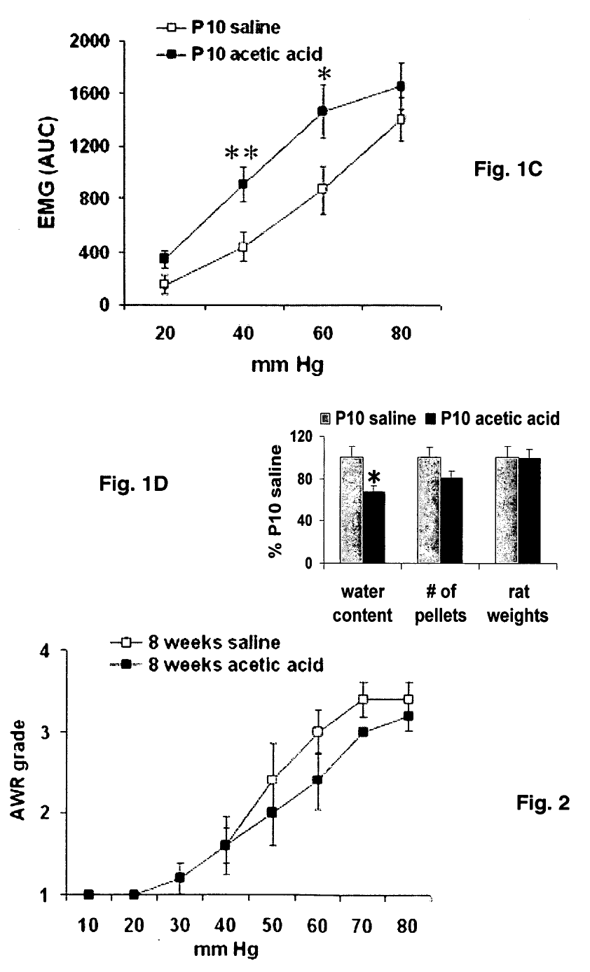

To determine whether a mild chemical irritation of the colon of neonatal rats with dilute acetic acid would produce visceral hyperalgesia in adults, we infused the colon of P10 rats with 0.5% acetic acid; control littermates received saline. Rats were tested at 8 weeks of age for sensitivity to colorectal distention. A significant increase in AWR grade in response to increasing colorectal distention pressure was observed in P10 acetic acid treated rats, p<0.001 and P10 saline treated controls, p<0.001 (FIG. 1A). P10 acetic acid treated rats exhibited higher mean AWR scores at all distention pressures tested compared to controls (FIG. 1A). These increases were statistically significant at 40 mm Hg, p=0.009, 50 mm Hg, p=0.023, 60 mm Hg, p=0.014 and 70 mm Hg, p=0.03. Significance was not reached at 10 mm Hg (p=0.404), 20 mm Hg (p=0.088), 30 mm Hg (p=0.081) or 80 mm Hg, (p=0.495). In a separate experiment, EMG activity, measured in response to graded colorectal distention, was significantly higher in P10 acetic acid treated rats compared to controls (two-way repeated measures ANOVA, neonatal treatment: F=5.288, p=0.030) at 40 mm Hg, p=0.010 and 60 mm Hg, p=0.035 (FIG. 1B). Taken together, these data showed that adult rats treated with acetic acid, as neonates were more sensitive to colorectal distention compared to controls suggesting that acetic acid treatment of the colon of P10 rats produced a persistent visceral hypersensitivity.

### EXAMPLE 12

### Acetic Acid Treatment of Colon of Adult Rats

Whether acetic acid treatment was effective in producing persistent sensitization when administered to adult rats was determined. Eight weeks old rats received 1 ml each of a 5% solution of acetic acid in saline into the descending colon; controls received saline. After 4 weeks, rats were tested for sensitivity to colorectal distention. No significant differences were observed in AWR grades between groups at any distention pressure tested: 50 mm Hg, p=0.690, 60 mm Hg, p=0.421, 70 mm Hg, p=0.310, 80 mm Hg, p=0.69 (FIG. 2). These data indicated that acetic acid treatment of adult rats did not produce long lasting sensitization.

### EXAMPLE 13

### Evaluation of Neonatal and Adult Colons for Inflammation

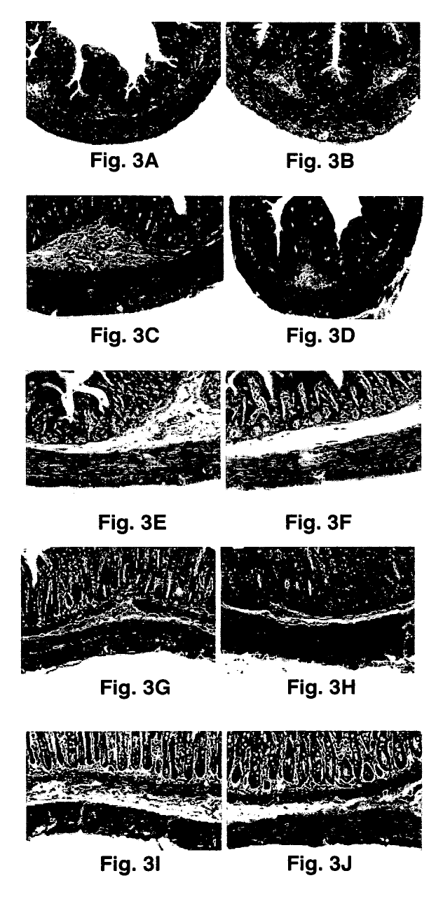

To rule out the possibility that the sensitizing effects of neonatal acetic acid exposure were due to inflammation/injury of the colon, the colons of P10 pups were examined. No histological signs of inflammation or tissue destruction were observed in the colons of pups treated with acetic acid or saline at 4 or 24 hours post treatment (FIG. 3A-D). Similarly, MPO values were not significantly different between the two groups at either 4 hrs, 24 SE 9 vs 13 SE 3, p=0.383, or 24 hours, 11 SE 3 vs 11 SE 2, p=0.875. These findings indicated that acetic acid treatment did not induce a significant inflammatory response in the colon of these pups.

To determine whether persistent hypersensitivity achieved in neonatal acetic acid treated rats was due to the development of a chronic colitis in adult, H&E stained sections of the colons of 8 weeks old adult rats were examined for histopathological signs of inflammation, myoloperoxidase (MPO) activity and inflammatory cytokine levels. Upon histopathological examination, no evidence of inflammation or abnormalities in structure was noted in both saline as well as acetic acid treated groups and no inflammatory infiltrates were observed (FIG. 3E-J). When MPO activity was measured in extracts from proximal, middle and distal colon, no significant increases in activity were detected in acetic acid treated rats (Table I). Likewise, no significant differences in the levels of several pro-inflammatory cytokines such as IL-1b, IL-1a, IL-2, IL-6, TNF-a or IFN-g were observed between saline and P10 AA treated rats in middle and distal colons (Table II). However in proximal colon, IFN-g was 1.5 fold higher in P10AA treated rats (p<0.01). Since mast cells can affect both motility and sensitivity, mast cells in proximal, middle and distal colon were counted. Mast cell counts (number of mast cells per high-powered field) were not significantly different between P10 saline and P10 acetic acid treated rats in proximal (8.3 SE 6.5 vs 6.3 SE 3.4, p=0.795), middle (18 SE 13 vs 7.7 SE 1.2, p=0.330) or distal colon (15.5 SE 0.4 vs 9.3 SE 4.1, p=0.325). Thus collectively, the data show that inflammation/abnormalities such as that are observed in typical inflammatory models was absent in this model ruling out their involvement in the persistent hypersensitivity. Thus a rat model of persistent visceral sensitization caused by neonatal exposure to a mild acid in the absence of ongoing inflammation is provided.

### EXAMPLE 14

### Neonatal Capsaicin Causes Persistent Sensitization

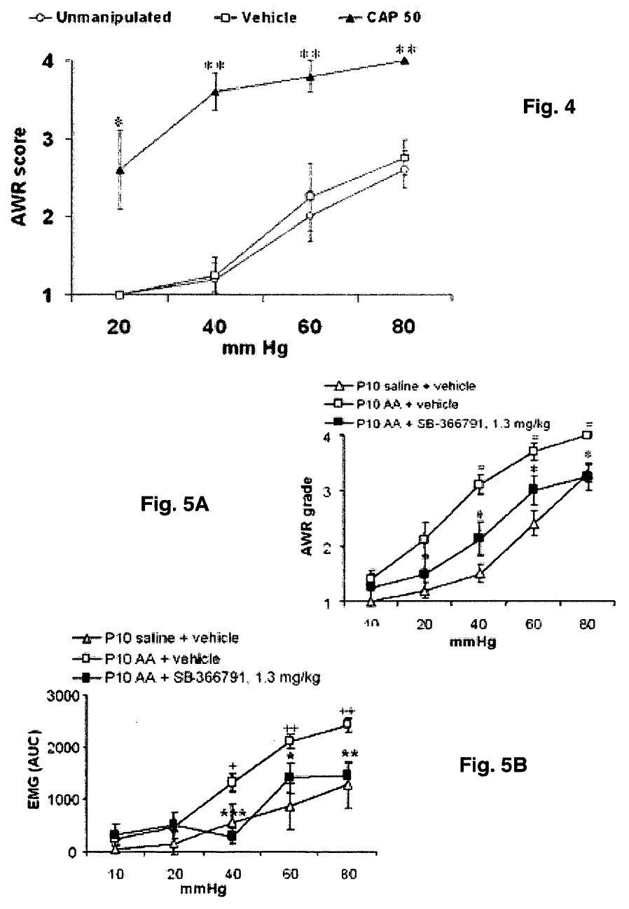

To examine the molecular events that may be involved in persistent sensitization it was hypothesized that the acid-responsive receptor, TRPV1 may initiate long lasting colonic sensitization. To this end, 10 days old rat pups were infused with 0.2 ml of a 50 mg/ml capsaicin solution or vehicle and tested them for sensitivity to colorectal distention at 7 weeks. Rats that received 50 mg/ml CAP as neonates were significantly more sensitive to colorectal distention compared to both unmanipulated and vehicle treated rats at 20 (p=0.007), 40 (p<0.001), 60 (p<0.001) and 80 (p<0.001) mm Hg. (FIG. 4).

### EXAMPLE 15

### Effect of TRPV1 Antagonism in Neonates

Ten days old rat pups received TRPV1 antagonist or vehicle subcutaneously 30 minutes prior to colon acetic acid infusion and were tested for sensitivity to colorectal distention at 8 weeks of age. Comparison of mean AWR scores at each distention pressure revealed significant differences at pressures of 40 (F=16.94, p<0.001), 60 (F=11.08, p<0.001) and 80 mm Hg (F=9.16, p=0.00111) (FIG. 5A). Post-hoc analysis showed significant differences between AWR scores of SB-366,791 and vehicle treated P10 acetic acid rats at these three distention pressures as well as between P10 acetic acid vehicle treated and P10 saline treated rats (p<0.05). Similarly, neonatal treatment resulted in significant differences in mean EMG activity (F=6.688, p=0.005), FIG. 5B. EMG activity of P10 acetic acid rats pre-treated with SB-366,791 was significantly less than P10 acetic acid vehicle treated controls at distention pressures of 40 (p=0.002), 60 (p=0.034) and 80 (p=0.004). P10 acetic acid rats pre-treated with vehicle displayed significantly higher EMG responses to colorectal distention compared to P10 saline vehicle at distention pressures of 40 (p=0.025), (60 p<0.001) and 80 (p<0.001) confirming that they were sensitized. Thus, P10 acetic acid rats pre-treated with SB-366,791 at a dose of 1.3 mg/kg showed significant decreases in sensitivity to colorectal distention at 40, 60 and 80 mm Hg both by AWR grade and by EMG compared to vehicle pre-treated P10 acetic acid rats.

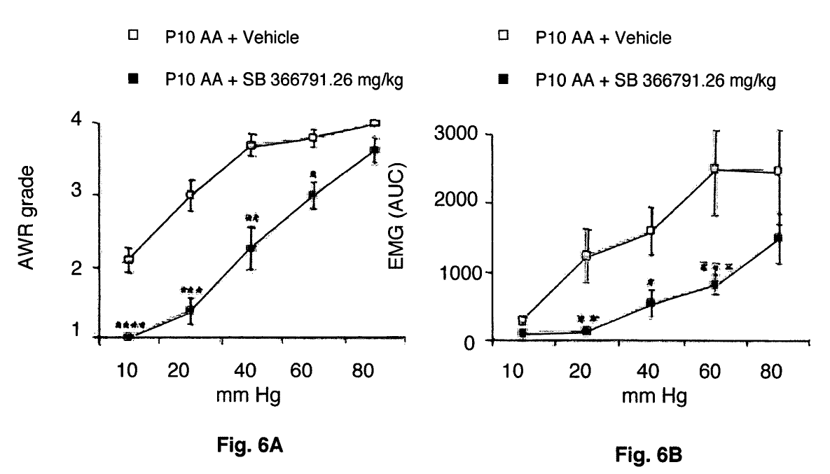

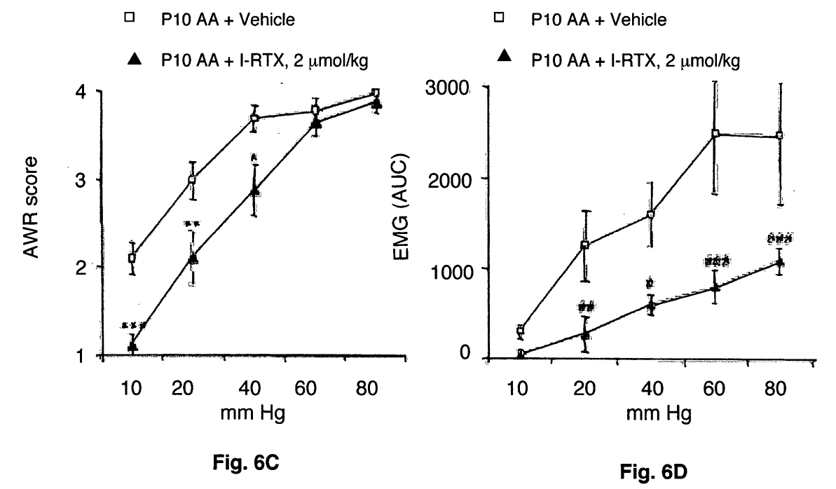

To confirm these results, SB-366791 and a second TRPV1 antagonist, I-RTX were tested in a second independent experiment. Neonatal pre-treatment with 2.6 mg/kg dose of SB-366,791 significantly decreased sensitivity of P10 acetic acid rats to colorectal distention at 10 (p=0.002), 20 (p=0.001), 40 (p=0.003) and 60 mm (p=0.014) Hg by AWR grade (FIG. 6A) and at 20 (p=0.038), 40 (p=0.050) and 60 (p=0.003) mm Hg by EMG (FIG. 6B). Neonatal pre-treatment with a 2 mg/kg dose of 1-RTX produced a significant decreased sensitivity of P10 acetic acid treated rats to colorectal distention as measured by AWR grade at 10 (p=0.002), 20 (p=0.037) and 40 (p=0.05) mm Hg (FIG. 6C) and by EMG at 20 (p=0.003), 40 (p=0.047), 60 (p<0.001) and 80 (p<0.001) mm Hg (FIG. 6D). Thus, antagonism of the TRPV1 prior to neonatal administration of acetic acid attenuated the development of sensitivity to colorectal distention.

### EXAMPLE 16

### Effect of TRPV1 Antagonism in Adults

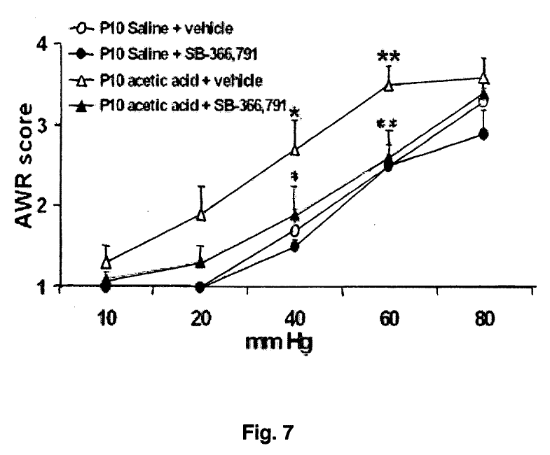

To determine whether TRPV1 antagonism reduced sensitivity to colorectal distention in adult rats that were neonatally treated with acetic acid or saline, adult rats were treated with 1.3 mg/kg i.p. SB-366791 or vehicle 30 minutes prior to graded colorectal distention. AWR scores of drug and vehicle treated rats as adults within each neonatal treatment group (P10 acetic acid or P10 saline) were compared at each distention pressure by Wilcoxon signed rank test. SB-366,791 treatment caused a significant decline in the mean responses of P10 acetic acid treated rats to colorectal distention at 40 mm Hg (p=0.023) and 60 mm Hg, (p=0.021) with a trend towards significance at 20 mm Hg (p=0.094) (FIG. 7). No significant differences were observed at 10 mm Hg, p=0.444 or 80 mm Hg, p=0.224. SB-366,791 treatment produced no significant effect upon the responses of P10 saline treated rats to colorectal distention (10, p=1.000, 20, p=1.000, 40, p=0.652, 60, p=1.000, 80, p=0.375). The AWR scores of vehicle treated P10 acetic acid rats were significantly greater than those of vehicle P10 saline rats at 40 and 60 mm Hg (p=0.042 and p=0.009) confirming that the P10 acetic acid treated rats were sensitized to colorectal distention.

### EXAMPLE 17

### TRPV1 Expression

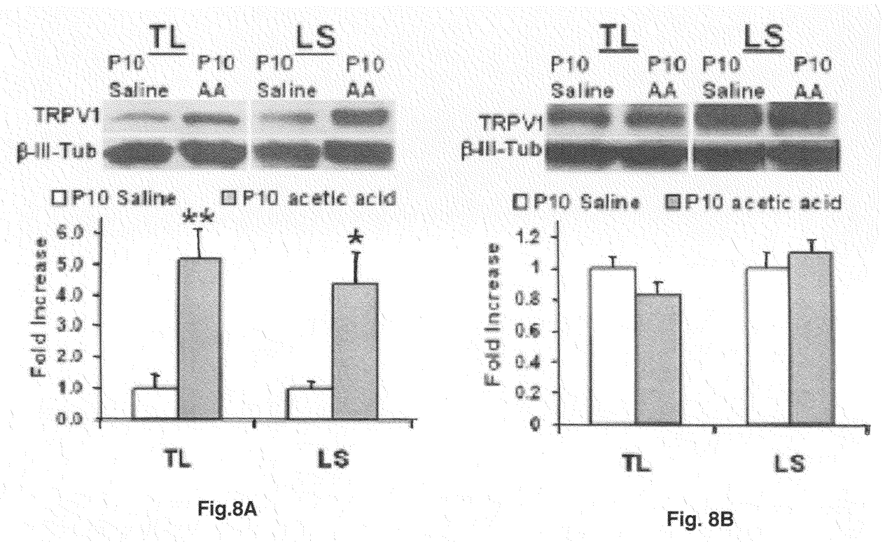

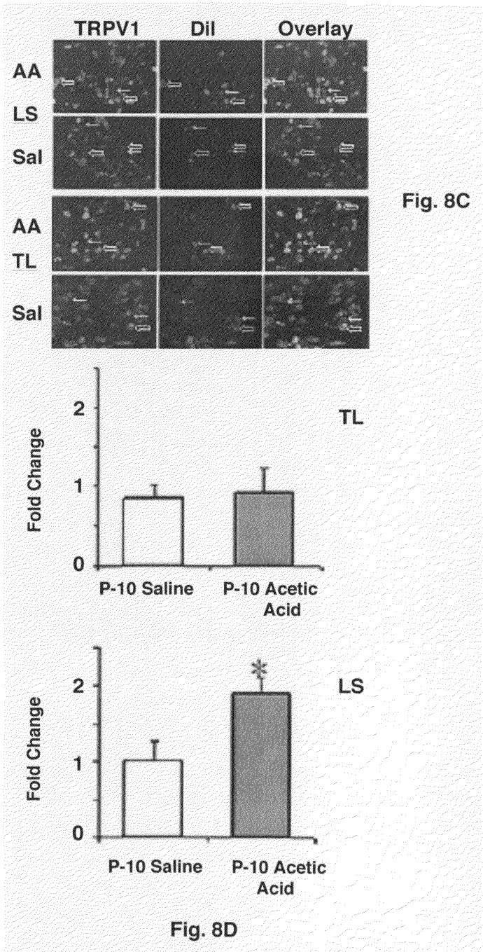

To determine whether TRPV1 expression was altered in adult rats that were sensitized with acetic acid as neonates, TRPV1 expression in DRG containing colon afferents and in the spinal cord was measured. TRPV1 protein expression, measured in whole DRG extracts was significantly increased in both TL (p<0.01) and LS DRG (p<0.03) (FIG. 8A) in rats with visceral hypersensitivity. No change in TRPV1 protein expression was observed in the spinal cord (FIG. 8B). There were significant increases in the number of colon DRG neurons identified by uptake of the retrograde label DiI that expressed TRPV1 immunoreactivity in both TL (40.6% to 67.3%, p<0.001) and LS DRG (38.8% to 54.0%, p=0.24) in rats with visceral hypersensitivity (FIG. 8C and Table III). TRPV1 mRNA was significantly increased 1.9 fold (p<0.05) in LS DRG, but no change was observed in TL DRG (FIG. 8D).

### EXAMPLE 18

### Increased Excitability of Colon DRG Neurons in Adult Rats Treated with Acetic Acid as Neonates

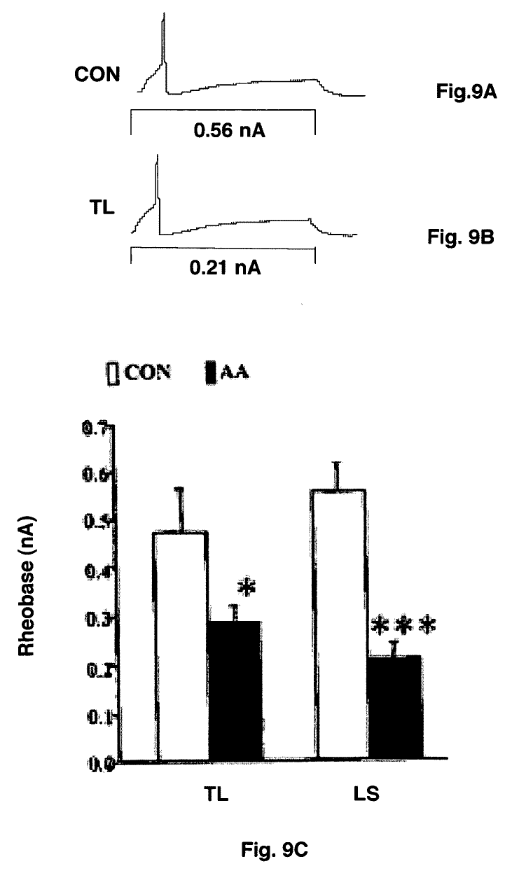

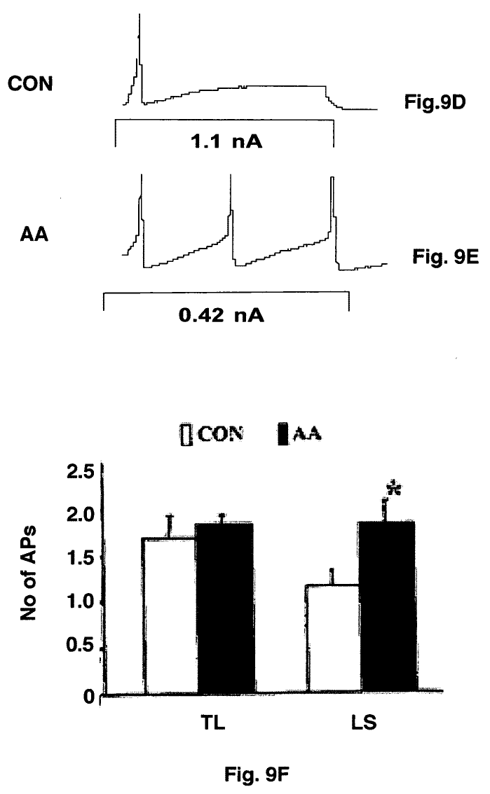

To determine whether the sensitivity of colon DRG neurons was altered in eight weeks old rats in this model, action potential threshold (rheobase) and firing frequency of acutely dissociated lumbar/sacral (LS), and thoracolumbar (TL) colon DRG neurons, identified by the presence of the retrograde label DiI, were measured in current clamp experiments. Both TL and LS colon DRG neurons isolated from P10 AA treated rats show a significant decline in average rheobase: LS, 0.56 to 0.21 nA, p=0.000128, TL, 0.47 to 0.29, p=0.034 (FIG. 9A). LS neurons from acetic acid treated rats also showed a significant increase in number of action potentials elicited by a current injection at 2× the rheobase, 1.2 to 1.8, p=0.0057 (FIG. 9B). Consistent with these results, recordings from both LS and TL nerve fibers in a similar model of neonatal irritation induced persistent visceral hypersensitivity and show increased firing rates of both pelvic and splanchnic nerve primary afferents in response to colorectal distention indicative of primary afferent sensitization [151]. Taken together, these data indicate that both LS and TL colon DRG neurons show increased excitability although differences appear to be greater in LS neurons. The robust differences observed will enable an assessment of the effects of prophylactic treatment with TRPV1 and MAPK inhibitors on the excitability of these neurons.

### EXAMPLE 19

### Increased Expression of TRPV1 mRNA in Lumbar/Sacral DRG is Associated with Persistent Sensitization

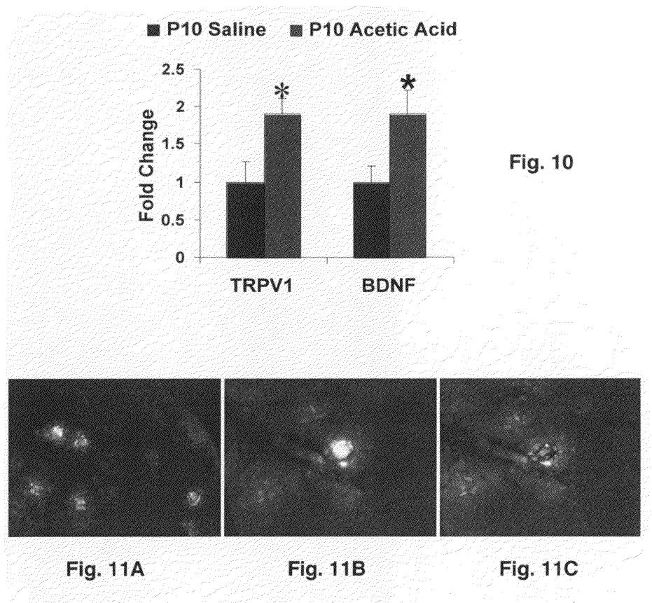

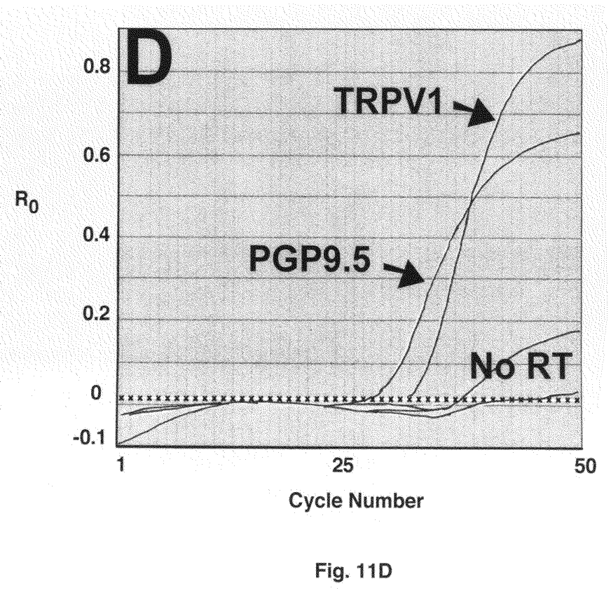

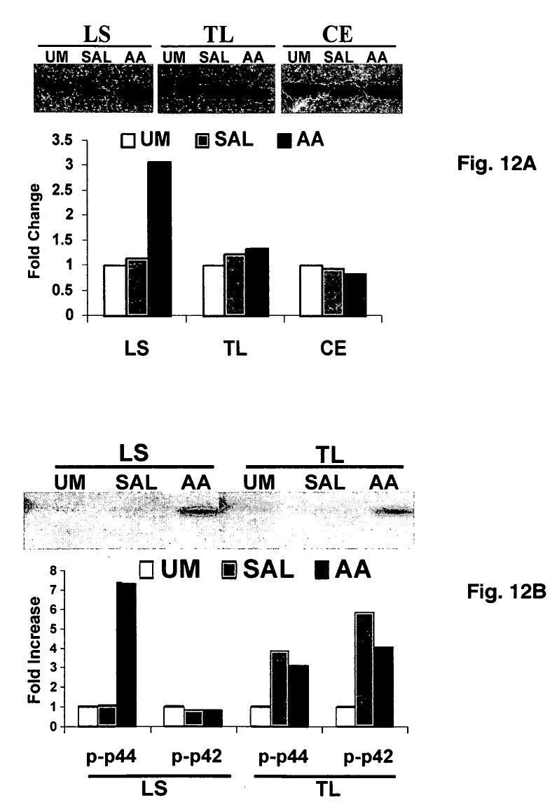

TRPV1 and BDNF mRNA expression was measured in S1 DRG isolated from adult rats that were treated with acetic acid or saline as neonates by quantitative RT-PCR. Both TRPV1 and BDNF expression were increased 1.9 fold in P10 acetic acid treated rats compared to P10 saline treated controls (FIG. 10). To increase the sensitivity and specificity of this gene expression assay, measurements will be made in colon afferent neurons identified by uptake of the retrograde label fluorogold and isolated by laser capture microdissection (LCM) (FIG. 11).

### EXAMPLE 20

### MAPK Activation in DRG Containing Colon Afferents

To determine whether acetic acid infusion of the colons of 10 days old pups activated MAPK in DRG containing soma of colon afferents, combined lumbar/sacral (LS) and thoracolumbar (TL) DRG from 4 pups/treatment group were assayed for p-p38 or ERK (p-p42/p-p44) content by western blot 1 hour after acetic acid infusion. Cervical (CE) DRG from the same animals were examined as an internal control for specificity of stimulation. A three-fold increase in p-p38 was observed in LS DRG in rat pups one hour after acetic acid infusion compared to saline and unmanipulated controls (FIG. 12A). No large changes in p-p38 were observed in either TL or CE DRG. There was a seven-fold increase in p-p44 in LS DRG from acetic acid treated pups, but no change was observed in p-p42 (FIG. 12B). In TL DRG, both p-ERK were increased 3-5 fold in both saline and acetic acid treated pups suggesting that ERK activation in TL DRG was due to the

The following references were cited herein:
1. Hammer & Talley, The American Journal of Medicine 1999; 107:5S-11S.
2. Drossman D. In: Rome II: the Functional Gastrointestinal disorders: diagnosis, pathophysiology, and treatment: a multinational consensus. McLean, Degnon Associates, 2000:764.
3. Lembo et al., Contemporary Diagnosis and Management of Irritable Bowel Syndrome. Handbooks in Healthcare Co., Newton, Pa., 2002:43-54.
4. Ritchie R. Gut 1973; 14:125-32.
5. Quigley E M. Gastroenterol Clin North Am. 2005; 34(2):221-33.
6. Schwetz I et al., Minerva Med 2004; 95:419-26.
7. Kellow J E et al., Gastroenterology 1990; 101:1621-1627.
8. Drossman D A et al., Ann Intern Med 1990; 113:828-833.
9. Drossman D A, et al. Ann Intern Med 1995; 123:782-794.
10. Talley N J et al. Gastroenterology 1994; 107:1040-1049.
11. Al-Chaer E D et al., Gastroenterology 2000; 119:1276-1285.
12. Coutinho S V et al. Am J Physiol 2002; 282:G307-G316.
13. Rosztoczy A et al., Neurogastroenterol Motil 2003:15:679-686.
14. Mayer E A et al., Gastroenterology 2002; 122: 2032-48.
15. Rong W et al., J. Physiol. 2004; 560(Pt 3):867-81.
16. Jones III R C W et al., J Neuroscience 2005; 25(47): 10981-10989.
17. Brierley S M et al., Gastroenterology 2004; 127:166-17.
18. Ward S M et al., J. Comp. Neurol. 2003; 465, 121-135.
19. Ness T J and Gebhart G F. Brain Research 1988; 450:153-169.
20. Ness T J et al., Pain 1990; 43:377-386.
21. Appleyard C B & Wallace J L Amer J Physiol 1995; 269:G119-G 125.
22. Bhatia M et al Proc Natl Acad Sci USA 1998; 95:4760-4756.
23. Gunthorpe M J et al., Neuropharmacology 2004; 46:133-149.
24. Rami H K et al. Med. Chem. Lett. 2004; 14:3631-3634.
25. Varga A et al., Neuroscience Letters 2005; 385:137-142.
26. Wahl P et al. Mol Pharmacol 2001; 59:9-15.
27. Jhaveri M D et al., Eur J of Neurosci 2005; 22:361-370.
28. Kindig et al., Am J Physiol Heart Circ Physiol 2005; 288:H1867-H1873.
29. Petho G et al., Pain 2004; 109:284-290.
30. Rigonis M et al., Br J. Pharmacol. 2003; 138(5):977-85.
31. Valenzano K J & Sun Q. Curr Med. Chem. 2004; 11(24):3185-202.
32. Whitehead W E et al., Gastroenterology 1990; 98:1187-1192.
33. Mertz H et al., Gastroenterology 1995; 109:40-52.
34. Munakata J et al., Gastroenterology 1997; 112:55-63.
35. Verne G N et al., Pain 2001; 93:7-14.
36. Drewes A M et al., Scand J Gastroenterol 2001; 36:827-32.
37. Naliboff B D et al., Gut 1997; 41:505-512
38. Burton M B & Gebhart G F. Brain Res 1995; 672:77-82.
39. La J H et al., World Journal of Gastroenterology; 2003; 9:2791-2795.
40. Chuang H-H et al., Nature 2001; 411:957-962.
41. Sugiuar T et al., J. Neurosci. 2004; 24(43):9521-30.
42. Stelt M et al., The EMBO Journal 2005; 24:3026-3037.
43. Moriyama T et al., J of Neuroscience 2003; 23(14):6058-6062.
44. Hwang S W et al., Proc Natl Acad Sci USA 2000; 97:6155-60.
45. Shin J et al., Proc Natl Acad Sci USA 2002; 99:10150-5.
46. Lin C et al., Brain Res 2003; 971:73-82.
47. Davis J B et al., Nature 2000; 405:183-187.
48. Caterina M J et al., Science 2000; 411:957-962.
49. Dinis, P. et al. J Neurosci 24, 11253-63 (2004).
50. Lima-Junior, R. C. et al., Planta Med 72, 34-9 (2006).
51. Maia, J. L. et al., Biol Pharm Bull 29, 82-5 (2006).
52. Ghilardi J R et al., J. Neurosci. 2005; 25(12):3126-31.
53. Schicho R et al., Eu J of Neurosci 2004; 19:1811-1818.
54. Fernihough, J et al., Neurosci Lett 388, 75-80 (2005).
55. Breese, N. M et al., Pain 115, 3749 (2005).
56. Ji, R. R et al., Neuron 36, 57-68 (2002).
57. Gopinath, P. et al. BMC Womens Health 5, 2 (2005).
58. Tympanidis, P. et al. Eur J Pain 8, 129-33 (2004).
59. Morgan, C. R et al., J Orofac Pain 19, 248-60 (2005).
60. Matthews, P. J. et al. Eur J Gastroenterol Hepatol 16, 897-902 (2004).
61. Chan, C. L. et al. Lancet 361, 385-91 (2003).
62. Domotor, A. et al. Inflammopharmacology 13, 161-77 (2005).
63. Yiangou, Y. et al. Lancet 357, 1338-9 (2001).
64. Chan C L et al., Lancet 2003; 361, 385-391.
65. Amadesi A et al., J Neurosci 2004; 24(18):4300-4312.
66. Sugiura T et al., J Neurosci 2004; 24(43):9521-9530.
67. Chadwick V et al., Gastroenterology 2002; 122:1778-1783.
68. Gonsalkorale W M et al., Gut 2003; 52:91-93.
69. Gwee K-A et al., Gut 2003; 52:523-526.
70. Collins S M et al., Am J. Gastroenterol. 2005; 100(11):2517-8
71. Barreau F et al., Gastroenterology. 2004; 127(2):524-34.
72. Barreau F et al., Gut 2004; 53:501-506.
73. Barbara G et al., Gastroenterology 2004; 126:693-702.
74. La J H et al., J Vet Sci. 2004; 5(4):319-24.
75. Barbara G et al., Neurogastroenterol Motil. 2006 January; 18(1):6-17.
76. Bradesi et al., Am J Physiol Gastrointest 2005 July; 289(1):G42-53.
77. Demaude J et al., Gut 2006; 55:655-661.
78. Ferrier L et al., Gastroenterology 2003; 125:795-804.
79. McRoberts J A et al., Gastroenterology 2001; 120:1737-1748.
80. Page A J et al., Gut 2005; 54; 1408-1415.
81. Wynn et al., Am J Physiol Gastrointest Liver 2004; 287:G647-G657.
82. Vlaskovska M et al., J. Neurosci. 2001; 21(15):5670-7.
83. Birder L A et al., Nature Neuroscience 2002; 5:856-860.
84. Fitzgerald M. Br. Med. Bull 1991, 47:667-675.
85. Fitzgerald M & Jennings E: Proc Natl Acad Sci USA 1999, 96:7719-7722.
86. Spiller R C. Gastroenterology 2003; 124:1662-1671.
87. Matthews P J et al, Eur J Gastroenterol Hepatol. 2004; 16(9):897-902.
88. Chan C L et al, Lancet 2003; 361, 385-391.
89. Yiangou Y et al, Lancet 2001; 57:1338-1339.
90. Ganju P et al, Eur J Pharm 2001; 421:191-199.
91. Averill S et al, Mol Cell Neurosci 2001; 18:183-96.
92. Aley K O et al, J Neurosci 2001; 21(17):6933-6939.
93. Ji R R et al, 2002; Neuron 36, 57-68.
94. Kim S Y et al, Neuroreport 2002; 13:2483-6.
95. Dai Y et al, J Neurosci 2002; 22:7737-7745.
96. Jin S X et al, J Neurosci 2003; 23(10):4017-22.
97. Obata K et al, J Neurosci 2003; 23:4117-4126.
98. Svensson C I et al, J Neurochem 2003; 86:1534-1544.
99. Scha″fers M et al, J Neurosci 2003; 23(7):2517-2521.
100. Obata K et al, Neuroscience 2004; 126 1011-1021.
101. Sweitzera S M et al, Pain 2004; 109:409-419.
102. Sweitzer S M et al, Pain. 2004; 111(3):278-85.
103. Dai Y et al, Pain 2004; 108:258-66.
104. Schicho R et al, Neuroscience. 2005; 134(2):505-14.
105. Mizushima T et al, Pain. 2005; 113(1-2):51-60.
106. Obata K et al, Eur J. Neurosci. 2004; 20(11):2881-95.
107. Ji R-R et al, Neurobiol Dis 2001; 8:1-10.
108. Chang L et al, Nature 2001; 410(6824):37-40.
109. Sweatt J D. J Neuroch, 2001; 76:1±10.
110. Pezet S et al, Brain Res Brain Res Rev 2002; 40:240-9
111. Michael G J et al, J Neurosci 1997; 17:8476-90.
112. Garraway S M et al, Eur J Neurosci 2003; 18:2467-76.
113. Delafoy L et al, Gut 2006; 55:940-945.
114. Howell S et al, Am J Gastroenterology 2005; 100:2071.
115. Ishiwata H et al, Neuroscience 2005; 133:893-901.
116. Papaioannou A et al, Neuroscience 2002; 114:195-206.

Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of retarding the development of hypersensitivity in an individual in need of such treatment comprising: administering to said individual a pharmacologically effective amount of a transient receptor potential vanilloid 1 (TRPV1) antagonist.

2. The method of claim 1, wherein said hypersensitivity is visceral or somatic.

3. The method of claim 2, wherein said visceral hypersensitivity is due to mechanical, chemical or thermal stimuli.

4. The method of claim 1, wherein said hypersensitivity is present in the absence of overt inflammation or peripheral pathology.

5. The method of claim 1, wherein said transient receptor potential vanilloid 1 antagonist is SB-366791.

6. The method of claim 1, wherein said transient receptor potential vanilloid 1 antagonist is I-RTX.

7. The method of claim 1, wherein said transient receptor potential vanilloid 1 antagonist attenuates excitability of colon DRG neurons.

8. The method of claim 7, wherein said attenuation of the excitability of colon DRG neurons is due to inhibition of the MAPK pathway.

9. The method of claim 1, wherein said individual suffers from the irritable bowel syndrome, functional dyspepsia, visceral hyperalgesia, visceral allodynia or altered gastric motility.

10. A method for ameliorating irritable bowel syndrome in an individual in need of such treatment comprising; administering a pharmacologically effective amount of a transient receptor potential vanilloid 1 (TRPV1) antagonist.

11. The method of claim 10, wherein said transient receptor potential vanilloid 1 antagonist is SB-366791.

12. The method of claim 10, wherein said transient receptor potential vanilloid 1 antagonist is I-RTX.

13. The method of claim 10, wherein said transient receptor potential vanilloid 1 antagonist attenuates excitability of colon DRG neurons.

14. The method of claim 13, wherein said attenuation of the excitability of colon DRG neurons is due to inhibition of the MAPK pathway.

15. The method of claim 9, wherein said individual suffers from visceral hyperalgesia, visceral allodynia, abdominal bloating and altered gut motility.

16. A method of reducing sensitization of the colon dorsal root ganglion neurons in an individual in need of such treatment comprising; administering a pharmacologically effective amount of a transient receptor potential vanilloid 1 (TRPV1) antagonist.

17. The method of claim 16, wherein said sensitization leads to the development of chronic visceral hypersensitivity.

18. The method of claim 17, wherein said chronic visceral hypersensitivity is present in the absence of any overt inflammation or peripheral pathology.

19. The method of claim 16, wherein said sensitization of the colon dorsal root ganglion neuron is due to early life environmental effects, stress, psychologic state and genetic factors.

20. The method of claim 16, wherein said transient receptor potential vanilloid 1 antagonist attenuates excitability of colon DRG neurons.

21. The method of claim 20, wherein said attenuation of the excitability of colon DRG neurons is due to inhibition of the MAPK pathway.

22. The method of claim 16, wherein said transient receptor potential vanilloid 1 antagonist is SB-366791.

23. The method of claim 16, wherein said transient receptor potential vanilloid 1 antagonist is I-RTX.

24. The method of claim 16, wherein said individual suffers from the irritable bowel syndrome, functional dyspepsia, visceral hyperalgesia, visceral allodynia or altered gastric motility.


SEQUENCE LISTING (6 sequences)

SEQ ID NO: 1 - DNA, 19 bp, artificial sequence
  primer_binding: forward primer for TRPV1
gcaagaagcgcctgactga

SEQ ID NO: 2 - DNA, 22 bp, artificial sequence
  primer_binding: reverse primer for TRPV1
tgagcatggcttttagcagaca

SEQ ID NO: 3 - DNA, 29 bp, artificial sequence
  probe sequence for TRPV1
tttcctgtctctgggtctttgaactcgct

SEQ ID NO: 4 - DNA, 21 bp, artificial sequence
  primer_binding: forward primer for beta-III-tubulin
gggcctttggacacctattca

SEQ ID NO: 5 - DNA, 18 bp, artificial sequence
  primer_binding: reverse primer for beta-III-tubulin
gccctttggcccagttgt

SEQ ID NO: 6 - DNA, 31 bp, artificial sequence
  probe sequence for beta-III-tubulin
cctgacaactttatcttcggtcagagtggtg